SEQUENCE LISTING (6 sequences)

SEQ ID NO: 1 - DNA, 19 bp, Mus musculus
aagaaccacagttgttgcg

SEQ ID NO: 2 - DNA, 19 bp, Mus musculus
gtaccgcacgtcattcgta

SEQ ID NO: 3 - DNA, 53 bp, Artificial Sequence
  Primer
aagaaccacagttgttgcgttcaagagacgcaacaactgtggttctttttttt

SEQ ID NO: 4 - DNA, 61 bp, Artificial Sequence
  Primer
aattaaaaaaaagaaccacagttgttgcgtctcttgaacgcaacaactgtggttcttggcc

SEQ ID NO: 5 - DNA, 53 bp, Artificial Sequence
  Primer
gtaccgcacgtcattcgtattcaagagatacgaatgacgtgcggtactttttt

SEQ ID NO: 6 - DNA, 61 bp, Artificial Sequence
  Primer
aattaaaaaagtaccgcacgtcattcgtatctcttgaatacgaatgacgtgcggtacggcc

(19) United States
(12) Patent Application Publication
(10) Pub. No.: US 2012/0115927 A1
(43) Pub. Date: May 10, 2012

(54) PHARMACEUTICAL AGENT FOR PREVENTING CELL DEATH

(75) Inventors: Futoshi Shibasaki, Tokyo (JP); Li Chen, Tokyo (JP)

(73) Assignee: TOKYO METROPOLITAN INSTITUTE OF MEDICAL SCIENCE, Tokyo (JP)

(21) Appl. No.: 13/259,351

(22) PCT Filed: Mar. 23, 2010
(86) PCT No.: PCT/JP2010/054988

§ 371 Date: Jan. 13, 2012

(30) Foreign Application Priority Data
Mar. 23, 2009 (JP) 2009-070529

(51) Int. Cl. A61K 31/713; A61P 9/10; A61P 25/00; A61P 7/00

(52) U.S. Cl. 514/44 A

(57) ABSTRACT

Disclosed is a pharmaceutical agent for preventing cell death. The occurrence of cell death can be prevented by inhibiting the function of Int6 protein in an affected area. Then, a pharmaceutical agent comprising a substance capable of inhibiting the function of Int6 protein is prepared. The pharmaceutical agent can be used for preventing cell death.

### CROSS-REFERENCE TO RELATED APPLICATIONS

This invention claims priority on Japanese Patent Application No. 2009-70529 filed on Mar. 23, 2009, which is herein incorporated by reference.

### TECHNICAL FIELD

This invention relates to pharmaceutical agents for preventing cell death.

### BACKGROUND ART

Cell death is deeply involved in various diseases including neurodegenerative diseases, ischemic disorders, and inflammatory diseases. Among these, cerebral infarction and myocardial infarction with the main symptom of cell death are serious diseases and are globally one of the main causes of human death (see, for example, N. Suwanwela and W. J. Koroshets, Acute ischemic stroke: overview of recent therapeutic developments. Annual Review of Medicine 58: 89-106 (2007)). Cells of brain and heart tissues are not regenerated once they are destructed, and patients often suffer from serious aftereffects even if they survived. For example, as aftereffects, motor, memory, and emotional disorders may remain in the case of the cerebral infarction, and reduction of heart pump function and arrhythmia may remain in the case of the myocardial infarction.

In the case of cerebral infarction and myocardial infarction, progress of the symptom, and as a consequence, improvement in the survival rate and alleviation of the infarction aftereffects of the patient will be realized if the cell death caused by the vascular disorder can be prevented within short period from the onset of the symptom (namely, in the “acute phase”).

A common surgical treatment performed for vascular disorder causing the cerebral infarction and the myocardial infarction is balloon angioplasty. In this treatment, a catheter is inserted in the blood vessel, and is expanded to dilate the clogged part of the blood vessel for recanalization. This balloon angioplasty requires special installation such as cardiac catheterization room. On the other hand, a common medicinal treatment is thrombolytic therapy in which the blood clot clogging in the blood vessel is dissolved by administering a thrombolytic agent (for example, tissue plasminogen activator such as alteplase) (see, for example, Japanese Patent Application Laid-Open No. 2008-230968, M. Fisher and B. Bastan, Treating acute ischemic stroke. Current Opinion in Pharmaceutical agent Discovery and Development 11: 626-625 (2008)). This therapy has been pointed to involve a risk of cerebral hemorrhage as a side effect.

### SUMMARY OF INVENTION

### Technical Problem

An object of the present invention is to provide pharmaceutical agents for preventing cell death.

### Solution to Problem

The inventors of the present invention found that progress of the myocardial infarction is suppressed when a siRNA against Int6 gene is administered to a myocardial infarction model mouse to thereby suppress expression of Int6 protein. The present invention has been completed on the bases of such finding.

In the present invention, the term “cell death” includes both “apoptosis” and “necrosis” in human and non-human mammals. The “prevention of cell death” means not only prior inhibition of occurrence of the cell death of a particular cell but also prior inhibition of progress of the cell death in a particular cell population.

The pharmaceutical agent of the present invention is a medicine for preventing cell death and contains a substance that suppresses function of Int6 protein. The substance that suppresses the function of Int6 protein may be a substance that suppresses expression of Int6 protein, and the substance that suppresses the expression of Int6 protein may be a siRNA against Int6 gene.

In the present invention, the cell death may have been induced by vascular disorder.

The pharmaceutical agent of the present invention may be a medicine for treating cerebral infarction or myocardial infarction.

Furthermore, the pharmaceutical agent of the present invention may be administered either before the occurrence of the cell death, within 6 hours from the onset of the vascular disorder or within 3 hours from the onset of the vascular disorder.

### BRIEF DESCRIPTION OF DRAWINGS

FIG. 1 shows pictures of the heart of animals, which were taken when two weeks have passed since they underwent a surgical operation inducing a vascular disorder and were administered with a negative control vector (A, B, and C) or a siRNA-Int6 vector (D, E, and F) in an Example of the present invention.

### DESCRIPTION OF EMBODIMENTS

Next, embodiments of the present invention completed by the finding as described above are described in detail by referring to Examples.

Unless otherwise noted, methods described in standard protocols, for example, J. Sambrook, E. F. Fritsch, and T. Maniatis (Ed.), Molecular cloning, a laboratory manual (3rd edition), Cold Spring Harbor Press, Cold Spring Harbor, N.Y. (2001); and F. M. Ausubel, R. Brent, R. E. Kingston, D. D. Moore, J. G. Seidman, J. A. Smith, and K. Struhl (Ed.), Current Protocols in Molecular Biology, John Wiley & Sons Ltd., as well as their modifications and improvements are used in the embodiments and Examples. When commercially available reagent kits and assay apparatus are used, protocols attached are used unless otherwise noted.

The objects, features, advantages, and ideas of the present invention are clear for those skilled in the art from the description of the invention, and those skilled in the art will be readily capable of reproducing the invention. The embodiments and Examples as described below are preferable embodiments of the present invention presented for the purpose of illustration and explanation, and the present invention are not limited by these embodiments and Examples. It is clear for those skilled in the art that the description of the present invention can be modified in various ways within the scope and intention of the invention herein described.

The pharmaceutical agent for preventing cell death according to the present invention (hereinafter also referred to as a cell death-preventing pharmaceutical agent) has a characteristic feature that it contains a substance that suppresses function of Int6 protein in the animal to which the pharmaceutical agent is administered.

The animal to which the cell death-preventing pharmaceutical agent is administered is not particularly limited. Preferable animals include human and non-human vertebrates, and the more preferable animal is human among them.

In the present invention, Int6 protein means human Int6 represented by Sequence No. 1 or its homolog including its ortholog. The homolog may be from non-limited animal species, and exemplary species include non-human vertebrates such as mouse, rat, chimpanzee, dog, avians such as chicken, and amphibians such as Xenopus laevis. Here, homology between the human Int6 protein and its homolog may vary with the species from which the homolog is derived. However, the homology is generally at least 50%, preferably at least 80%, more preferably at least 90%, and most preferably at least 95%. The Int6 protein and the homolog may share common a function. In addition, the Int6 gene according to the present invention is not particularly limited as long as it is a gene coding for the Int6 protein described in the present invention.

The substance which suppresses the function of Int6 protein is not particularly limited as long as the substance lowers the function of the Int6 protein of the animal, to which the substance is administered, totally at the cell level, and examples include low molecular weight compounds which suppress the function of the protein itself, and dominant negative protein mutants such as Int6ΔC (Int6 mutant protein wherein 119, 185, or 295 amino acids from the C terminal have been deleted) (see Chen et al., Journal of Biological Chemistry 282: 12707-16, 2007). Examples also include substances which suppress the function of the Int6 protein as a result of suppressing the expression of the protein; preferred is the use of a substance that causes the complete loss of the function of the Int6 protein, and in particular, a substance which suppresses the expression of the Int6 protein. This expression-suppressing substance is not particularly limited as long as it is a substance capable of suppressing the expression of the Int6 protein. Such substances may include, for example, an antisense nucleic acid for a transcription product of the Int6 gene or its part, a nucleic acid having ribozyme activity for a transcription product of the Int6 gene, and miRNA, shRNA, and miRNA for the Int6 gene. The methods for using such nucleic acid are commonly known to those skilled in the art, who can also readily design the nucleic acids used in such methods.

These nucleic acids may be chemically synthesized, processed into a dosage form and used. For example, in the case of RNA, an expression vector suitable for expression of the RNA may be prepared, processed into a dosage form, and then administered to a patient so that the RNA should be expressed in the body of the patient.

The cell death which is to be prevented by the cell death-preventing pharmaceutical agent is not particularly limited for the tissue where it occurs and its cause. However, the cell death caused by a vascular disorder is preferable and the cell death caused with cerebral infarction or myocardial infarction is more preferable.

The cell death-preventing pharmaceutical agent may be prepared into a dosage form by using various pharmaceutically acceptable pharmaceutical additives known to those skilled in the art such as carrier, diluent, and excipient. The dosage form is not particularly limited as long as the pharmaceutical agent can reach the lesion in the body of the patient. Exemplary dosage forms include oral preparations such as tablet, capsule, granule, powder, syrup, enteric coated tablet, controlled-release capsule, cashew, chewable tablet, drops, pill, peroral liquid preparation, candy tablet, controlled-release tablet, and controlled-release granules as well as injections. In addition to the pharmaceutical additives as described above, the pharmaceutical agent of the present invention may also have a different pharmaceutical composition.

The cell death-preventing pharmaceutical agent may be administered at a necessary dose within established safety limit to a human or other mammal by an appropriate method. The dosage of the pharmaceutical agent of the present invention may be appropriately determined by considering dosage form, administration method, age and weight of the patient, symptoms of the patient, and the like, and finally according to the judgment of a physician or a veterinarian. Any appropriate administration method may be used for delivering the pharmaceutical agent of the dosage form to the lesion, and the preferred is parenteral administration such as local injection or coating to or on the lesion tissue and systemic administration by intravenous, subcutaneous, intramuscular, intraperitoneal, or other injection.

The cell death-preventing pharmaceutical agent is preferably administered before the occurrence of cell death of the target cell in the target animal to which the pharmaceutical agent is administered. In the present invention, the term “administration” means completion of the administration of the amount effective for preventing the cell death, and “before the occurrence of cell death of the target cell” includes not only the case in which the cell death has not at all occurred in the patient's tissue but also the case in which cell death has already occurred in a part of the tissue. In the latter case, progress of cell death can be suppressed totally in the tissue by preventing expansion of the cell death to the healthy cells around the cells that have already undergone the cell death. When the cell death is induced by vascular disorder, the pharmaceutical agent of the present invention is preferably administered in the acute phase of the vascular disorder; for example, preferably within 6 hours, and more preferably within 3 hours from the onset of the vascular disorder.

### Examples

This Example shows that occurrence of the infarction can be prevented and progress of the symptoms of the myocardial infarction can be suppressed by suppressing expression of Int6 protein with a siRNA against Int6 gene.

[Preparation of an Expression Vector Having siRNA Against Int6 Gene]

siRNA against Int6 and siRNA for negative control having the nucleotide sequences as shown below were synthesized by using Silencer siRNA Construction Kit (Ambion), and conducing PCR with the primers as described below and the mouse genomic DNA for the template. The resulting siRNA was inserted in pSilencer 1.0-U6 vector (Ambion) to prepare an expression vector having the siRNA against mouse Int6 gene (mouse-siRNA-Int6 vector) and an expression vector having the negative control siRNA, respectively.

Mouse siRNA-Int6:
(SEQ ID NO: 1)
AAGAACCACAGTTGTTGCG
Negative control siRNA:
(SEQ ID NO: 2)
GTACCGCACGTCATTCGTA
Primers used:
Mouse siRNA-Int6:
5′-primer (SEQ ID NO: 3):
AAGAACCACAGTTGTTGCGTTCAAGAGACGCAACAACTGTGGTTCTTTT
TTTT
3′-primer (SEQ ID NO: 4):
AATTAAAAAAAAGAACCACAGTTGTTGCGTCTCTTGAACGCAACAACTG
TGGTTCTTGGCC
Negative control siRNA:
5′-primer (SEQ ID NO: 5):
GTACCGCACGTCATTCGTATTCAAGAGATACGAATGACGTGCGGTACTT
TTTT
3′-primer (SEQ ID NO: 6):
AATTAAAAAAGTACCGCACGTCATTCGTATCTCTTGAATACGAATGACG
TGCGGTACGGCC

[Suppression of Mouse Myocardial Infarction by Int6-siRNA]

A mouse (8 week C57B6 male, about 25 g (body weight)) was anaesthetized with 50% oxygen+50% laughing gas+4% isoflurane, and after intubation, the mouse was artificially respirated with 50% oxygen+50% laughing gas+2% isoflurane. The amount of ventilation per ventilation was 0.7 ml and the number of ventilation was 120. Under this general anesthesia, chest was opened between left ribs, specifically between fifth and sixth ribs, and anterior descending coronary artery was ligated to induce cardiovascular disorder. Myocardial infarction was thus induced in the tissue where the blood flow was stopped by this cardiovascular disorder.

The vector prepared as above was dissolved in physiological saline at a concentration of 10 μg/μl. 200 μg solution of the siRNA-Int6 vector or the negative control vector was administered to cardiac muscle on the peripheral side of the ligated blood vessel in the heart of the mouse in which the vascular disorder had been induced. Then the chest was stitched, and breeding of the mice was continued after their recovery.

Two weeks after the operation, the heart was taken out from the mice, and frozen-stored. Frozen sections were then prepared and mounted on polylysine coated glass slides (manufactured by Matsunami Glass Ind., Ltd.). The sections were fixed with 3% formalin, and after staining with hematoxylin and eosin, the state of the myocardial infarction were microscopically observed on the sections.

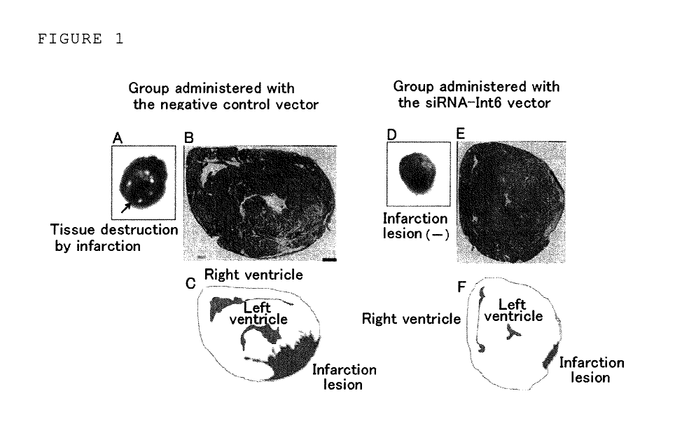

As shown in FIG. 1, among the animals in which the vascular disorder had been induced with the ligation, the size of the myocardial infarction lesions of animals administered with the siRNA-Int6 vector was smaller compared to that of the animals administered with the negative control siRNA vector. Thus, the siRNA-Int6 vector is capable of suppressing the cell death associated with the myocardial infarction and supressing progress of the myocardial infarction.

### INDUSTRIAL APPLICABILITY

The present invention provides pharmaceutical agents for preventing cell death.

1. A method for suppressing cell death in a patient, comprising administering to the patient a substance that suppresses function of Int6 protein.

2. The method according to claim 1, wherein the substance suppresses expression of the Int6 protein.

3. The method according to claim 2 wherein the substance is a siRNA against Int6 gene.

4. The method according to claim 1, wherein the cell death is induced by a vascular disorder.

5. The method according to claim 4, wherein the vascular disorder is due to cerebral infarction or myocardial infarction.

6. The method according to claim 1, wherein the substance is administered before occurrence of the cell death.

7. The method according to claim 4, wherein the substance is administered within 6 hours after occurrence of the vascular disorder.

8. The method according to claim 4, wherein the substance is administered within 3 hours after occurrence of the vascular disorder.

9. A method for suppressing cell death in a patient, comprising suppressing function of Int6 protein in the patient.

10. The method according to claim 9, wherein expression of the Int6 protein is suppressed.

11. The method according to claim 10 wherein the expression is suppressed with a siRNA against Int6 gene.

12. The method according to claim 9, wherein the cell death is induced by a vascular disorder.

13. The method according to claim 12, wherein the vascular disorder is due to cerebral infarction or myocardial infarction.

14. The method according to claim 9, wherein the function of Int6 protein is suppressed before occurrence of the cell death.

15. The method according to claim 12, wherein the function of Int6 protein is suppressed within 6 hours after occurrence of the vascular disorder.

16. The method according to claim 12, wherein the function of Int6 protein is suppressed within 3 hours after occurrence of the vascular disorder.
